(19) United States
(12) Patent Application Publication
(10) Pub. No.: US 2016/0120477 A1
(43) Pub. Date: May 5, 2016

(54) BIOLOGICAL-INFORMATION PROCESSING APPARATUS AND BIOLOGICAL-INFORMATION PROCESSING METHOD

(71) Applicant: Seiko Epson Corporation, Shinjuku-ku (JP)

(72) Inventor: Yusuke Takahashi, Matsumoto-shi (JP)

(21) Appl. No.: 14/991,650

(22) Filed: Jan. 8, 2016

Related U.S. Application Data
(63) Continuation of application No. PCT/JP2014/003652, filed on Jul. 9, 2014.

(30) Foreign Application Priority Data
Jul. 12, 2013 (JP) 2013-146148

(51) Int. Cl. A61B 5/00; A61B 5/024

(52) U.S. Cl. CPC A61B 5/7207; A61B 5/7246; A61B 5/7225; A61B 5/681; A61B 5/02438

(57) ABSTRACT

A biological-information processing apparatus includes a plurality of filters that detects a biological signal including a pulse wave component and a body motion noise component, detects a body motion signal correlated to the body motion noise component, and separates the pulse wave component and the body motion noise component included in the biological signal on the basis of the body motion signal. The filters have different filter characteristics. The biological-information processing apparatus calculates, for each of the filters, a correlation coefficient between the body motion signal and at least one of the pulse wave component and the body motion noise component and determines, on the basis of the correlation coefficient, as a pulse wave signal, the pulse wave component separated using any one filter.

### CROSS-REFERENCES TO RELATED APPLICATIONS

This application is a continuation of PCT Application PCT/JP2014/003652, filed Jul. 9, 2014, which is a PCT Application of Japanese Patent Application No. 2013-146148, filed Jul. 12, 2013, the entireties of which are hereby incorporated by reference.

### BACKGROUND

1. Technical Field

The present invention relates to a biological-information processing apparatus and a biological-information processing method for measuring a pulse rate of a subject.

2. Background Art

There has been known a pulsimeter worn on an arm or the like of a subject to measure a pulse rate during exercise such as walking or running. The pulsimeter includes a pulse wave sensor. The pulsimeter detects a change in a blood flow rate of the subject and acquires a biological signal. The pulsimeter extracts a signal component (a pulse wave component) equivalent to a pulse wave from the biological signal and calculates a pulse rate. Besides the pulse wave component, a body motion component due to a body motion during exercise of the subject is superimposed on the biological signal. Therefore, the pulsimeter further includes an acceleration sensor. The pulsimeter detects a body motion signal of the subject, estimates a body motion component from the body motion signal, and extracts a main pulse wave component. In order to estimate the body motion component, for example, a pulsimeter described in PTL 1 applies FFT (Fast Fourier Transform) processing to the biological signal and the body motion signal, estimates a frequency component corresponding to the body motion signal from a frequency component of the biological signal, and selects a frequency component equivalent to the pulse rate. A signal extracting apparatus described in PTL 2 estimates the body motion component from the body motion signal using an adaptive filter configured by an FIR (Finite Impulse Response) filter, reduces the body motion component from the biological signal, and extracts the main pulse wave component.

### CITATION LIST

### Patent Literature

PTL 1: JP-A-7-227383

PTL 2: JP-A-11-276448

### SUMMARY OF INVENTION

### Technical Problem

However, in the apparatuses described in PTL 1 and PTL 2, in a situation involving a change in the pulse rate and fluctuation in the body motion such as time of a sudden start of exercise by the subject, many noise components due to the body motion sometimes remain in an extracted estimated pulse wave component. Depending on a degree of the remaining noise components, the noise components affect specifying of a frequency component equivalent to the pulse rate. Therefore, further improvement is necessary. Specifically, in PTL 1, when the subject suddenly performs a rigorous motion, for example, at the start of exercise, the motion involves an increase in the pulse rate of the subject. Therefore, the frequency component equivalent to the pulse rate disperses. The dispersed frequency component of the pulse rate is mixed in a frequency component of the noise components due to the body motion. Therefore, it is difficult to specify the pulse rate. It is necessary to estimate the pulse rate using even information other than the biological signal and the body motion signal. In view of such a situation, there is a need for a contrivance to extract an estimated pulse wave component in which the remaining of the noise components included in the biological signal is further reduced. In PTL 2, in a series of scenes in which an exercise state changes at a sudden start of exercise by the subject and during the exercise after the start, many noise components sometimes remain partially in an estimated pulse wave component extracted by the adaptive filter. In a part where many noise components remain, the noise components affect specifying of the frequency component equivalent to the pulse rate. Therefore, further improvement is necessary.

In this way, even if there are changes of various exercise states of the subject, in order to more accurately measure the pulse rate of the subject, it is necessary to extract an estimated pulse wave component with noise components sufficiently attenuated.

### Solution to Problem

The present invention has been devised in order to solve at least a part of the problems and can be realized as the following forms or application examples.

### APPLICATION EXAMPLE 1

A biological-information processing apparatus according to this application example includes: a biological-signal detecting unit that detects a biological signal including a pulse wave component and a body motion noise component; a body-motion-signal detecting unit that detects a body motion signal; and a body-motion-noise removing unit that separates the pulse wave component and the body motion noise component from the biological signal on the basis of the body motion signal. The body-motion-noise removing unit includes: a plurality of filter units having different learning characteristics; a correlation-information calculating unit that calculates correlation information indicating a correlation degree between the body motion signal and output signals from the plurality of filter units; and a selecting unit that selects the output signal from the plurality of filter units on the basis of the correlation information.

According to this application example, since the body-motion-noise removing unit includes the plurality of filter units having the different learning characteristics, it is possible to obtain a plurality of output signals corresponding to scenes of various exercise states of a subject. The plurality of output signals are compared on the basis of the body motion signal and the correlation information. The body motion noise component having a high correlation degree with the body motion signal is calculated out of the plurality of output signals. It is possible to select the output signal including the pulse wave component with the body motion noise component sufficiently attenuated. That is, even if there are changes in various exercise states, it is possible to extract the pulse wave component with the body motion noise component sufficiently attenuated.

### APPLICATION EXAMPLE 2

It is preferable that the learning characteristics are varied according to a step size for controlling a following characteristic to fluctuation in the body motion signal.

According to this application example, since the learning characteristics include the step size, the plurality of filter units having the different learning characteristics respectively have different following characteristics corresponding to the fluctuation in the body motion signal. Therefore, it is possible to select the pulse wave component with the body motion noise component attenuated that is calculated most following the fluctuation in the body motion signal in the various exercise states of the subject.

### APPLICATION EXAMPLE 3

It is preferable that the correlation-information calculating unit calculates, for each of the output signals from the filter units, the correlation information on the basis of the body motion signal, and the selecting unit selects the output signal from the filter unit in which the absolute value of the correlation information is the smallest.

According to this application example, the selecting unit can select the output signal having the lowest correlation with the body motion signal. Therefore, the selected output signal is the output signal (an estimated pulse wave component) estimated as including least remaining of the body motion noise component.

### APPLICATION EXAMPLE 4

It is preferable that the output signals from the filter units are estimated pulse wave signals for estimating the pulse wave component.

### APPLICATION EXAMPLE 5

It is preferable that the correlation-information calculating unit calculates the correlation information on the basis of each of the output signals from the filter units and the body motion signal, and the selecting unit selects the output signal from the filter unit in which the absolute value of the correlation information is the largest.

According to this application example, the selecting unit can select the output signal representing a correlation closest to the body motion signal. Therefore, the selected output signal is the body motion signal and a signal estimated as most imitating noise involved in the body motion signal (an estimated body motion noise component). The body motion noise component can be attenuated most in the pulse wave component obtained by separating the body motion noise component from the biological signal. Therefore, it is possible to select the pulse wave component with the body motion noise component sufficiently attenuated.

### APPLICATION EXAMPLE 6

It is preferable that the output signals from the filter units are estimated body motion noise signals for estimating the body motion noise component.

### APPLICATION EXAMPLE 7

It is preferable that, when a difference between the correlation information of the filter unit that outputs the selected output signal and the correlation information of the other filter units exceeds a predetermined threshold, the selecting unit sets, as the learning characteristics of the other filters, the learning characteristics of the filter units that outputs the selected output signal.

According to this application example, by setting, in the other filters that do not produce results, the learning characteristics of the filter that appropriately attenuates the estimated body motion noise component from the biological signal, it is possible to increase filter performances to an appropriate level halfway in a filter operation. That is, the plurality of filters have even performance from a point in time when the learning characteristics are set. Thereafter, it is possible to perform adaptive processing (learning processing) based on the respective learning characteristics. Therefore, since the performances of the respective plurality of filters having the different learning characteristics are improved, features of the learning characteristics are directly reflected on the output signals. It is possible to extract the estimated pulse wave component with the estimated body motion noise component more precisely attenuated.

### APPLICATION EXAMPLE 8

It is preferable that the body motion signal includes acceleration signals in one axial direction or at least two axial directions crossing each other, and the signals from the axes are sequentially applied as the body motion signal.

According to this application example, by applying the body motion signals such as the acceleration signals in the axial directions superimposed on the biological signal to the filters one by one, it is possible to attenuate the noise component related to the superimposed signals. Therefore, it is possible to attenuate, for each of the signals, the noise component superimposed on the biological signal. It is possible to extract the estimated pulse wave component with less noise component.

### APPLICATION EXAMPLE 9

The body motion signal may include a contact pressure signal indicating pressing of a detection part of the biological signal.

According to this application example, by applying, to the filters, the body motion signal such as the contact pressure signal indicating the pressing of the detection part further superimposed on the biological signal, it is possible to attenuate the noise component due to, for example, a change in a wearing state of the biological-information processing apparatus worn on an arm or the like of the subject.

### APPLICATION EXAMPLE 10

The biological-information processing apparatus may further include a control unit that calculates a pulse rate on the basis of the signal selected by the selecting unit.

According to this application example, since the selected signal is the estimated pulse wave component with the noise component attenuated, in pulse rate calculation processing by FFT processing or the like, it is easy to specify a frequency indicating a pulse. It is possible to provide pulse rate calculation with high reliability. Time required for calculation is reduced. It is possible to suppress power consumption.

### APPLICATION EXAMPLE 11

A biological-information processing method according to this application example includes: a biological-signal detecting step for detecting a biological signal including a pulse wave component and a body motion noise component; a body-motion-signal detecting step for detecting a body motion signal; a body-motion-noise removal processing step for separating the pulse wave component and the body motion noise component from the biological signal on the basis of the body motion signal using a plurality of filter steps for separating the pulse wave component and the body motion noise component, the plurality of filter steps having different learning characteristics; a correlation-information calculating step for calculating correlation information indicating a correlation degree between the body motion signal and output signals from the plurality of filter steps; and a selecting step for selecting the output signal from the plurality of filter units on the basis of the correlation information.

According to this application example, since the biological-information processing method includes the plurality of filters having the different learning characteristics, it is possible to calculate body motion noise components and pulse wave components based on the respective learning characteristics. It is possible to select the output signal with less body motion noise component out of the output signals from the plurality of filter steps on the basis of the correlation information between the body motion signal and the output signal. Even in a portion with many changes in exercise states, the selected output signal is a signal with least remaining body motion noise component among the output signals calculated in the plurality of filter steps. Therefore, it is possible to extract the pulse wave component with the noise component sufficiently removed even if there are changes in various exercise states of a subject.

### BRIEF DESCRIPTION OF THE DRAWINGS

FIG. 1 is a front view of a pulsimeter.

FIG. 2(a) is a rear view of the pulsimeter and FIG. 2(b) is a use state view of the pulsimeter.

FIG. 3 is an explanatory diagram of the operation of a pulse wave sensor.

FIG. 4 is a block diagram showing an example of the functional configuration of the pulsimeter.

FIG. 5 is a block diagram showing an example of the functional configuration of a body-motion-noise removing unit.

FIG. 6 is a principle configuration block diagram of an adaptive filter.

FIG. 7 is an application example of the adaptive filter (at the start time of exercise).

FIG. 8 is an application example of the adaptive filter (at the time of stable exercise).

FIG. 9 is a flowchart for explaining a flow of a control program of the pulsimeter.

FIG. 10 is a flowchart for explaining a flow of body-motion-noise-component removal processing.

FIG. 11 is a graph showing an example of a calculated pulse rate.

FIG. 12 is a flowchart for explaining a flow of body-motion-noise-component removal processing in a second embodiment.

### DESCRIPTION OF EMBODIMENTS

Embodiments of the present invention are explained below with reference to the drawings. Note that, in the figures referred to below, scales of layers and members are set different from actual scales in order to show the layers and the members in recognizable sizes.

### First Embodiment

### (Exterior Configuration of a Biological-Information Processing Apparatus)

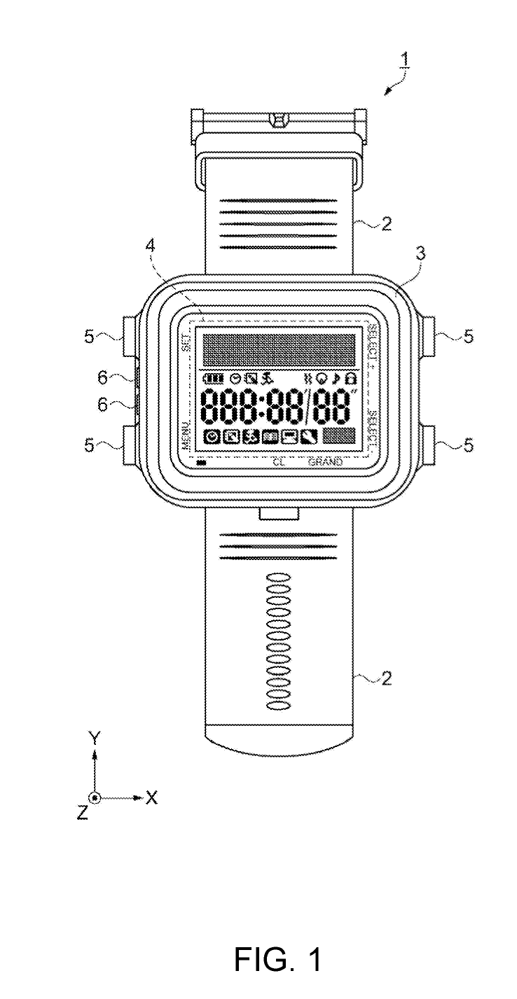

FIG. 1 is a front view of a pulsimeter in this embodiment. A pulsimeter 1 functioning as a biological-information processing apparatus includes a wristband 2. On a case 3, a display panel 4 for displaying time, an operation state of the pulsimeter 1, and various kinds of biological information (a pulse rate, exercise intensity, calorie consumption, etc.) using characters, numbers, icons, and the like is disposed.

Operation buttons 5 for operating the pulsimeter 1 are disposed in the peripheral portion (the side surfaces) of the case 3. The pulsimeter 1 operates using, for example, a built-in secondary battery as a power supply. On a side surface of the case 3, charging terminals 6 connected to an external charger to charge the built-in secondary battery are disposed.

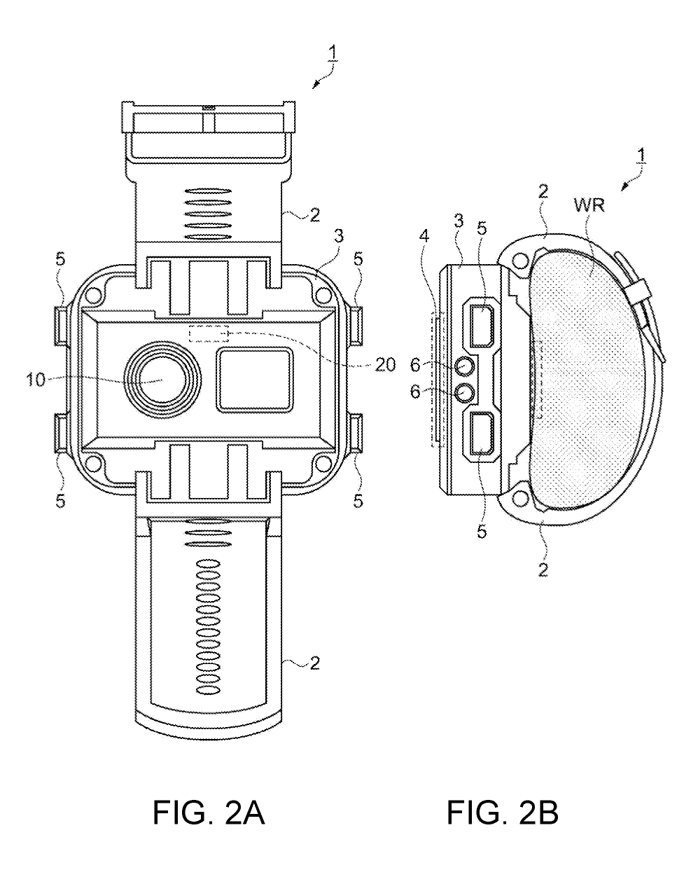

FIG. 2(a) is a rear view of the pulsimeter 1 and shows an exterior view of the pulsimeter 1 viewed from the back of the case 3. FIG. 2(b) is a use state view of the pulsimeter 1 and shows a side view of the pulsimeter 1 in a state in which the pulsimeter 1 is worn on a wrist WR of a subject.

On the rear surface of the case 3, a pulse wave sensor 10 that detects a change in a blood flow in a subcutaneous tissue (a shallow part) in the wrist WR or the like of the subject and outputs a biological signal is disposed. As a preferred example, the pulse wave sensor 10 is a photoelectric pulse wave sensor and includes a mechanism for optically detecting a change in a blood flow rate.

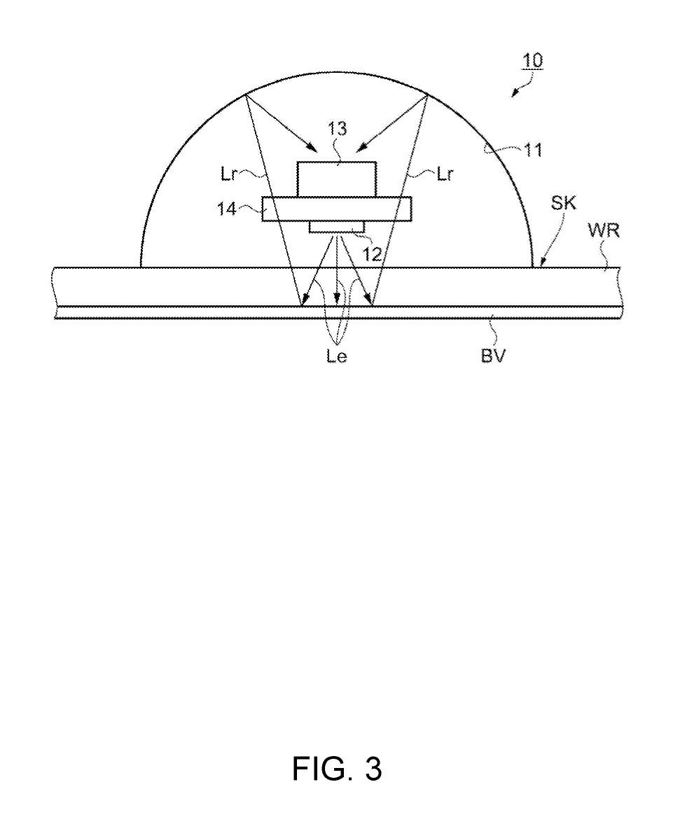

FIG. 3 is an explanatory diagram of the structure of the pulse wave sensor 10 and is an enlarged view of the internal structure of the pulse wave sensor 10 viewed from the side of the case 3. The pulse wave sensor 10 is disposed in a hemispherical housing space formed on the back side of the case 3 and having a circular bottom surface. In the housing space, a light emitting element 12 such as an LED (Light Emitting Diode) and a light receiving element 13 such as a photo transistor are incorporated. The inner surface of the hemisphere is a mirror-finished reflection surface 11. When an opening surface side of the hemisphere is regarded as a downward direction, the light receiving element 13 and the light emitting element 12 are respectively mounted on the upper surface and the lower surface of a substrate 14.

When light Le is irradiated toward a skin SK of the wrist WR of the subject by the light emitting element 12, a part of the irradiated light Le is reflected on a blood vessel BV under the skin and returns into the hemisphere as reflected light Lr. The reflected light Lr is further reflected on the hemispherical reflection surface 11 and made incident on the light receiving element 13 from above.

Reflected light intensity of the reflected light Lr from the blood vessel BV fluctuates reflecting a change in a blood flow rate according to light absorbing action of hemoglobin in the blood. The pulse wave sensor 10 causes the light emitting element 12 to blink at a predetermined cycle higher than the cycle of a beat. The light receiving element 13 receives the reflected light Lr in every lighting opportunity of the light emitting element 12, performs photoelectric conversion corresponding to the intensity of the received light, and outputs a biological signal as a signal of the change in the blood flow rate. The pulse wave sensor 10 causes the light emitting element 12 to blink at a frequency of, for example, 128 Hz.

As shown in FIG. 2(a), the pulsimeter 1 incorporates an acceleration sensor 20 for detecting a body motion of the subject. As shown in FIG. 1, the acceleration sensor 20 is, for example, an acceleration sensor having directions of three axes; a Z axis positive in the normal direction of a cover glass surface of the case 3, i.e., the display surface side, a Y axis in the up-down direction positive in a 12 o'clock direction of a clock, and an X axis in the left-right direction positive in a 3 o'clock direction of the clock.

In a state in which the pulsimeter 1 is worn on the arm, the X axis coincides with a direction from the elbow to the wrist of the subject. The acceleration sensor 20 detects accelerations in the three axes of the X axis, the Y axis, and the Z axis and sequentially outputs at least the accelerations in the X axis and the Y axis as body motion signals. The pulsimeter 1 detects, on the basis of the body motion signals detected by the acceleration sensor 20, body motions during various kinds of exercise including cyclical body motions (e.g., a pitch and movement of the arm) of the subject involved in walking and running.

Note that the acceleration sensor 20 is a sensor including acceleration sensors of the three axes. However, the acceleration sensor 20 only has to be a sensor including at least acceleration sensors of two axes. The acceleration sensor 20 may include acceleration sensors of substantially orthogonal two axes or may include acceleration sensors of three-dimensionally crossing multiple axes.

### (Functional Configuration of the Pulsimeter)

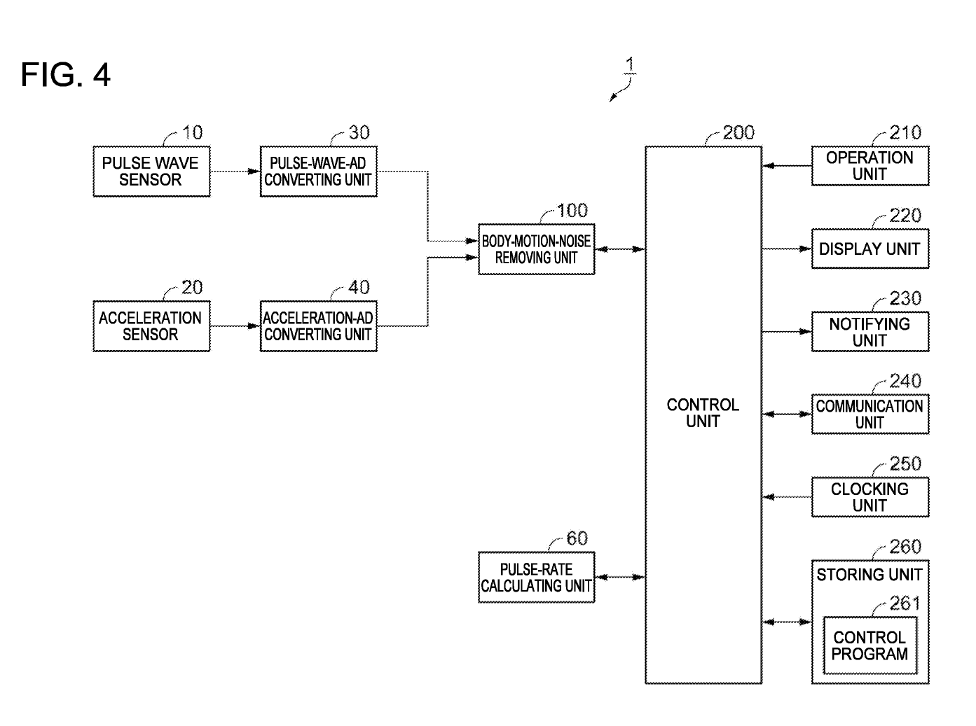

FIG. 4 is a block diagram showing an example of the functional configuration of the pulsimeter.

The pulsimeter 1 is configured from the pulse wave sensor 10, the acceleration sensor 20, a pulse-wave-AD converting unit 30, an acceleration-AD converting unit 40, a pulse-rate calculating unit 60, a body-motion-noise removing unit 100, a control unit 200, an operation unit 210, a display unit 220, a notifying unit 230, a communication unit 240, a clocking unit 250, a storing unit 260, and the like.

As explained above, the pulse wave sensor 10 is a sensor that measures a change in a blood flow rate of the subject wearing the pulsimeter 1. The pulse wave sensor 10 detects, as a biological signal, a volume change caused by inflow of a blood flow into a body tissue and outputs the biological signal amplified at a predetermined gain to the pulse wave sensor 10.

The pulse-wave-AD converting unit 30 samples the amplified biological signal of an analog form at a predetermined sampling time interval and converts the analog biological signal into a digital signal having predetermined resolution. The pulse-wave-AD converting unit 30 outputs the biological signal converted into the digital signal to the body-motion-noise removing unit 100.

As explained above, the acceleration sensor 20 is a sensor for grasping a body motion of the subject wearing the pulsimeter 1. The acceleration sensor 20 detects acceleration signals in the three axial directions of the X axis, the Y axis, and the Z axis as body motion signals. The body motion signals are amplified at a predetermined gain and output to the acceleration-AD converting unit 40.

The acceleration-AD converting unit 40 samples the amplified body motion signals of an analog form at a predetermined sampling time interval and converts the analog body motion signals into digital signals having predetermined resolution. The acceleration-AD converting unit 40 outputs the body motion signals converted into the digital signals to the body-motion-noise removing unit 100.

Note that the pulse wave sensor 10 and the pulse-wave-AD converting unit 30 are equivalent to the biological-signal detecting unit. The acceleration sensor 20 and the acceleration-AD converting unit 40 are equivalent to the body-motion-signal detecting unit.

The body-motion-noise removing unit 100 is a filter circuit that receives a biological signal and a body motion signal as inputs and separates a pulse wave component and a body motion noise component included in the biological signal. The filter circuit calculates an estimated body motion noise component from the body motion signal using an adaptive filter. Thereafter, the estimated body motion noise component is attenuated from the biological signal, whereby an estimated pulse wave component is extracted. The adaptive filter includes two kinds of adaptive filters having different learning characteristics and outputs, respectively from the adaptive filters, as output signals, the estimated body motion noise component and the estimated pulse wave component obtained by attenuating the estimated body motion noise component from the biological signal. Note that details of the body-motion-noise removing unit 100 are explained below.

The control unit 200 is a processor such as a MPU (Micro Processing Unit) or a DSP (Digital Signal Processor). The control unit 200 controls, on the basis of a control program 261 stored in the storing unit 260, units including the pulse wave sensor 10, the acceleration sensor 20, the body-motion-noise removing unit 100, the operation unit 210, and the display unit 220 configuring the pulsimeter 1.

The pulse-rate calculating unit 60 is a functional unit that calculates a pulse rate from the estimated pulse wave component output by the body-motion-noise removing unit 100. The pulse-rate calculating unit 60 is an assumed part obtained by regarding, as a constituent part, a function realized by a part of the control program 261 executed by the control unit 200. For example, the pulse-rate calculating unit 60 applies frequency resolution processing (FFT processing) to the estimated pulse wave component, analyzes signal intensity values of frequencies, and specifies a frequency spectrum equivalent to a pulse wave. The pulse-rate calculating unit 60 calculates a pulse rate from the frequency of the frequency spectrum of the pulse wave. In the pulse-rate calculating unit 60, in a process of the FFT processing, it is easier to specify a frequency that assumes a pulse if noise is less in the input estimated pulse wave component.

Note that, although not shown in the figure, the pulsimeter 1 may also include, as a functional unit, a calculating unit that calculates a pitch (steps per minute) for grasping an exercise state of the subject using a body motion signal, exercise intensity (MET) and a consumed calorie calculated using the pulse rate or the pitch, and the like.

The operation unit 210 is an input device including button switches and the like. The operation unit 210 outputs a signal of a depressed button to the control unit 200. Various instruction inputs such as a measurement instruction for a beat rate are performed by the operation of the operation unit 210. The operation unit 210 is equivalent to the operation buttons 5 shown in FIG. 1. Note that the configuration of the operation unit 210 is not limited to this and only has to be a configuration capable of performing a plurality of operation inputs. The display panel 4 may include a touch panel function.

The display unit 220 is a display device including an LCD (Liquid Crystal Display). The display device performs various kinds of display based on display signals input from the control unit 200. Various kinds of biological information (a pulse rate, exercise intensity, a consumed calorie, etc.) are displayed on the display unit 220. The display unit 220 is equivalent to the display panel 4 shown in FIG. 1.

The notifying unit 230 is a notifying device including a speaker and a piezoelectric oscillator. The notifying device performs various kinds of notification based on notification signals input from the control unit 200. For example, the notifying unit 230 performs various kinds of notification to the subject by causing the speaker to output alarm sound or vibrating the piezoelectric oscillator.

The communication unit 240 is a communication device for transmitting and receiving, according to the control by the control unit 200, information used inside the apparatus to and from an external information processing apparatus such as a PC (Personal Computer). As a communication system of the communication unit 240, it is possible to apply various systems such as a form for connecting the communication unit 240 by wire via a cable conforming to a predetermined communication standard, a form for connecting the communication unit 240 via an intermediate device also used as a charger called cradle, and a form for connecting the communication unit 240 by radio using a short-range radio communication.

The clocking unit 250 includes a quartz oscillator configured by a quartz oscillator and an oscillation circuit. The clocking unit 250 includes a clock function, a stopwatch function, and a clocking function such as generation of a sampling time for biological information and body motion information detection of the pulsimeter 1. Clocked time of the clocking unit 250 is output to the control unit 200 at any time.

The storing unit 260 is configured by a storage device such as a ROM (Read Only Memory), a flash ROM, or a RAM (Random Access Memory). The storing unit 260 has stored therein the control program 261 for the pulsimeter 1 and various programs, data, and the like for realizing various functions such as a function of the pulse-rate calculating unit 60, an exercise intensity calculating function, and a consumed calorie calculating function. The storing unit 260 includes a work area for temporarily storing data under processing, processing result, and the like of various kinds of processing.

### (Configuration of the Body-Motion-Noise Removing Unit)

Details of the body-motion-noise removing unit 100 are explained.

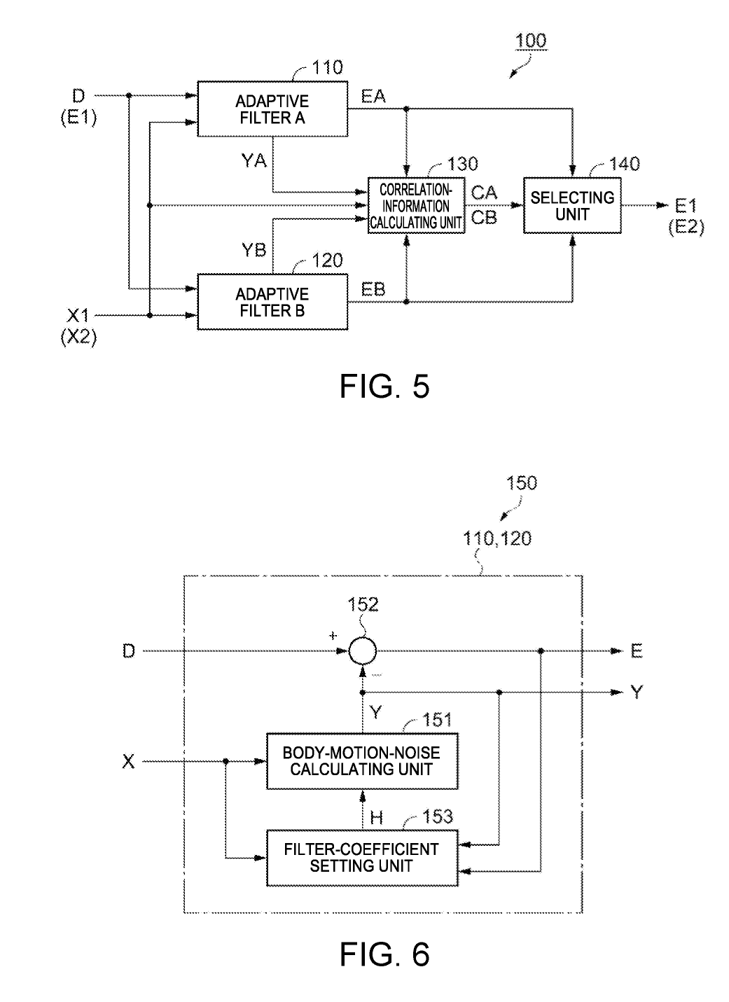

FIG. 5 is a block diagram showing an example of the functional configuration of the body-motion-noise removing unit.

The body-motion-noise removing unit 100 is configured from an adaptive filter A 110, an adaptive filter B 120, a correlation-information calculating unit 130, a selecting unit 140, and the like. The adaptive filter A 110 and the adaptive filter B 120 are equivalent to the plurality of filter units.

In the body-motion-noise removing unit 100, a biological signal D and a first axis signal X1 serving as a body motion signal are received as input signals. An estimated pulse wave component E1 is output. The first axis signal X1 is, for example, an acceleration signal in the X-axial direction. The estimated pulse wave component E1 is a signal component with noise due to acceleration in the X-axial direction attenuated. Further, in the body-motion-noise removing unit 100, the estimated pulse wave component E1 and a second axis signal X2 serving as a body motion signal are received as input signals. An estimated pulse wave component E2 is output. The second axis signal X2 is, for example, acceleration in the Y-axial direction. The estimated pulse wave component E2 is a signal component with noise due to accelerations in the X-axial direction and the Y-axial direction attenuated. Details are explained below.

The adaptive filter A 110 and the adaptive filter B 120 are filters having different learning characteristics of adaptive algorithms.

In the adaptive filter A 110, the biological signal D and the first axis signal X1 are input. An estimated body motion noise component YA is calculated while adapting learning characteristics explained below. A difference between the biological signal D and the estimated body motion noise component YA is output as an estimated pulse wave component EA.

In the adaptive filter B 120, the biological signal D and the first axis signal X1 are input. An estimated body motion noise component YB is calculated on the basis of learning characteristics different from the learning characteristics of the adaptive filter A 110. A difference between the biological signal D and the estimated body motion noise component YB is output as an estimated pulse wave component EB.

Note that the estimated body motion noise component YA and the estimated pulse wave component EA output from the adaptive filter A 110 and the estimated body motion noise component YB and the estimated pulse wave component EB output from the adaptive filter B 120 are equivalent to the output signals from the plurality of filter units.

In the correlation-information calculating unit 130, the first axis signal X1, the estimated pulse wave component EA, and the estimated pulse wave component EB are input. A correlation coefficient CA indicating a correlation degree of the first axis signal X1 and the pulse wave component EA and a correlation coefficient CB indicating a correlation degree of the first axis signal X1 and the estimated pulse wave component EB are respectively calculated and output to the selecting unit 140. Note that the correlation coefficient CA and the correlation coefficient CB are equivalent to the correlation information. Details of a portion where the estimated body motion noise component YA and the estimated body component noise component YB are input to the correlation-information calculating unit 130 shown in FIG. 5 are explained in a second embodiment.

In the selecting unit 140, the estimated pulse wave component EA, the estimated pulse wave component EB, the correlation coefficient CA, and the correlation coefficient CB are input. The estimated pulse wave component EA or the estimated pulse wave component EB is output as the estimated pulse wave component E1 on the basis of a comparison result of the correlation coefficient CA and the correlation coefficient CB. Details of the calculation of the correlation coefficients and determination of the comparison are explained below.

The estimated pulse wave component E1 output by the selecting unit 140 is the estimated pulse wave component EA or EB with the estimated body motion noise component YA or YB, which has a correlation with the first axis signal X1 superimposed on the biological signal D, attenuated. For example, in the X-axial direction, the first axis signal X1 is an acceleration signal generated in a direction from the elbow to the wrist in a state in which the subject is performing exercise such as running wearing the pulsimeter 1 on the wrist. Therefore, the estimated pulse wave component E1 is an estimated pulse wave component with an estimated body motion noise component generated in the direction from the elbow to the wrist attenuated. The movement of the arm in the running or the like is not only a linear movement. Therefore, a change in a body motion in the Y-axial direction is also large in addition to the X-axial direction. Therefore, body motion noise components in a plurality of directions are superimposed on the estimated pulse wave component E1. It is necessary to reduce the body motion noise component in the Y-axial direction from the estimated pulse wave component E1.

Therefore, the body-motion-noise removing unit 100 further reduces a body motion noise component having a correlation with the second axis signal X2 remaining in the estimated pulse wave component E1. The second axis signal X2 is, for example, a signal in the Y-axial direction. The estimated pulse wave component E1 and the second axis signal X2 are received as input signals. The estimated body motion noise components YA and YB concerning the second axis signal X2 are calculated from the estimated pulse wave component E1 in the adaptive filter A 110 and the adaptive filter B 120. The estimated pulse wave components EA and EB with the estimated body motion noise components YA and YB, which have a correlation with the second axis signal X2 remaining in the estimated pulse wave component E1, reduced are calculated. The estimated pulse wave component E2 selected by the selecting unit 140 using the correlation coefficient CA and the correlation coefficient CB calculated in the correlation-information calculating unit 130 is output. The estimated pulse wave component E2 is a signal including, as a main component, a pulse wave component obtained by removing the body motion noise components in the directions of the first axis signal X1 and the second axis signal X2 from the body motion signal D. The estimated pulse wave component E2 is output as a pulse wave signal from the body-motion-noise removing unit 100.

In this way, the noise removal processing by the adaptive filters disposed in parallel is sequentially executed on the basis of a plurality of acceleration signals. Consequently, it is possible to further improve performance for attenuating a body motion noise component.

### (Configuration of the Adaptive Filters)

Details of the adaptive filter A 110 and the adaptive filter B 120 are explained with reference to FIG. 6.

FIG. 6 is a principle configuration block diagram of an adaptive filter.

An adaptive filter 150 is a principle configuration of the adaptive filter A 110 and the adaptive filter B 120. The adaptive filter 150 is configured from a body-motion-noise calculating unit 151, a subtracting unit 152, a filter-coefficient setting unit 153, and the like. The adaptive filter 150 is a filter that separates an estimated pulse wave component E and an estimated body motion noise component Y while updating a filter coefficient H on the basis of the estimated pulse wave component E, the estimated body motion noise component Y, a body motion signal X, and the like. In the body-motion-noise calculating unit 151, the body motion signal X and the filter coefficient H are subjected to a product-sum operation and the body motion noise component Y is calculated. In the subtracting unit 152, the body motion noise component Y is subtracted from the biological signal D and the pulse wave component E is output. In the filter-coefficient setting unit 153, the filter coefficient H is calculated from the pulse wave component E, the biological noise component Y, and the body motion signal X and output to the body-motion-noise calculating unit 151.

### (Arithmetic Processing of the Adaptive Filter)

Arithmetic processing of the adaptive filter is explained.

The biological signal D and the body motion signal X are discrete data arranged in time series detected at a predetermined sampling cycle. A data sequence of the biological signal D is represented by d(i) and a data sequence of the body motion signal X is represented by x(i). A data sequence of the calculated body motion noise component Y is represented by y(i), a data sequence of the pulse wave component E is represented by e(i), and a data sequence of the filter coefficient H is represented by h(i). An argument i is a value used as an argument of a data sequence. The data sequence is data arranged in time series. Therefore, as the argument i is larger, the data is later in time. A maximum value of the argument i is the number of data accumulated during a predetermined period. Data having the same value of the argument i is data detected at substantially the same timing and data calculated at substantially the same timing. Data having an argument i−1 is data earlier in time than the argument i and is a sample value earlier by one sample. When the body motion noise component Y is explained as an example, data up to L samples before y(i) are y(i−1), y(i−2), . . . , and y(i−L).

Formulas configuring the adaptive filter 150 are explained using the data sequences.

Formula (1) is a formula for calculating a body motion noise component y(i) of an i-th argument. A product of a filter coefficient h(k) and a body motion signal x(i−k) is added up L times while increasing k from 1 to L. The body motion signal x(i−k) is a body motion signal up to L samples before the body motion signal x(i). L is equal to a filter length in the adaptive filter and is a tap of a filter.

Y(i)=Σ[h(k)−x(i−k)] k=1 to L Formula (1)

A Formula (2) is a formula for calculating a pulse wave component e(i) of the i-th argument. It is possible to calculate the pulse wave component e(i) by subtracting y(i) calculated by Formula (1) from the biological signal d(i).

e(i)=d(i)−y(i) Formula (2)

### (Learning Characteristics of the Adaptive Filter)

A formula (3) is a formula for updating the filter coefficient h(k). Already calculated values of variables of the right side are substituted in h(k) of the right side to update the filter coefficient h(k). The right side is calculated by multiplying together a step size μ, the pulse wave component e(i), and the body motion noise component y(i−k) and adding a product to the filter coefficient h(k). For example, when a value of the filter coefficient h(k) of the right side is not decided, a value set in advance or the like is set. The step size μ is explained below.

h(k)=h(k)+μ·e(i)·y(i−k) k=1 to L Formula (3)

The updated filter coefficient h(k) is substituted in Formula (1) in order to calculate the next body motion noise component y(i+1). The filter coefficient h(k) calculated last in the predetermined period is set as a value of the filter coefficient h(k) in the next predetermined period. However, when an output result of the adaptive filter is required by the determination of the calculated correlation coefficient value, the filter coefficient h(k) of the other adaptive filter may be set as a value of the filter coefficient h(k) in the next predetermined period. Note that the filter coefficient h(k) is equivalent to the learning characteristics of the filter.

In this way, L filter coefficients of h(l) to h(L) are updated. The updated filter coefficient h(k) is a coefficient for determining the learning characteristics of the filter and is a coefficient that determines whether the body motion noise component Y following fluctuation in the body motion signal X can be generated.

As seen in the right side of Formula (3), the step size μ is a parameter that determines the filter coefficient h(k) and may be a fixed value or may be calculated by an expression like Formula (4) described below. By adjusting the magnitude of a value of the step size μ, it is possible to relatively control following performance to a change of the body motion signal X and attenuation performance of noise. That is, when the step size μ is set to a large value, the adaptive filter is a filter having high following performance to a change in the body motion signal X. On the other hand, attenuation performance of noise decreases. When the step size μ is set to a small value, the adaptive filter is a filter having high attenuation performance of noise. On the other hand, the following performance to a change in the body motion signal X decreases. In the adaptive filter A 110 and the adaptive filter B 120, the step size μ is set to values of different magnitudes.

The step size μ may be calculated as indicated by Formula (4). The step size μ is a value obtained by dividing a fixed value α by a value obtained by adding up a square sum of the body motion signal x(i−k) and a fixed value β. By changing values of the fixed value α and the fixed value β, the magnitude of a numerical value of the step size μ can be adjusted.

μ=α/[β+Σx²(i−k)] k=1 to L Formula (4)

Dependency on a body motion signal of the step size μ is absorbed by normalizing the step size μ with power of the body motion signal.

When Formula (4) is used, the adaptive filter A 110 and the adaptive filter B 120 respectively retain the fixed value α and the fixed value β. Different values are set in the adaptive filter A 110 and the adaptive filter B 120.

In this way, the different step sizes μ are set in the adaptive filter A 110 and the adaptive filter B 120. Consequently, the body motion noise component y(i) is also calculated as different information through Formula (3) and Formula (1). Specifically, if the numerical value of the step size μ increases, for example, when cyclicity of exercise suddenly changes, the calculated body motion noise component y(i) tends to be capable of following more quickly to a frequency characteristic corresponding to an exercise cycle after the change. That is, following performance to a body motion signal that rises in a short time is high. Conversely, if the numerical value of the step size μ decreases, for example, when the cyclicity of exercise is stable, the calculated body motion noise component y(i) tends to be sufficiently attenuated in the pulse wave component e(i) calculated by Formula (2) by estimating the body motion signal x(i) and a response component of the body motion signal x(i). That is, attenuation performance of a noise component to a body motion signal with stable cyclicity of exercise is high.

In this way, it is possible to construct the adaptive filter having learning characteristics capable of controlling following performance and attenuation performance. Note that a relation between the numerical value of the step size μ and the tendency of the signal component applied with the adaptive filter is derived by, on the basis of a theoretical hypothesis, analyzing experiment data obtained by actually repeating various exercise states of a plurality of subjects.

### (Action of the Adaptive Filter and the Learning Characteristics)

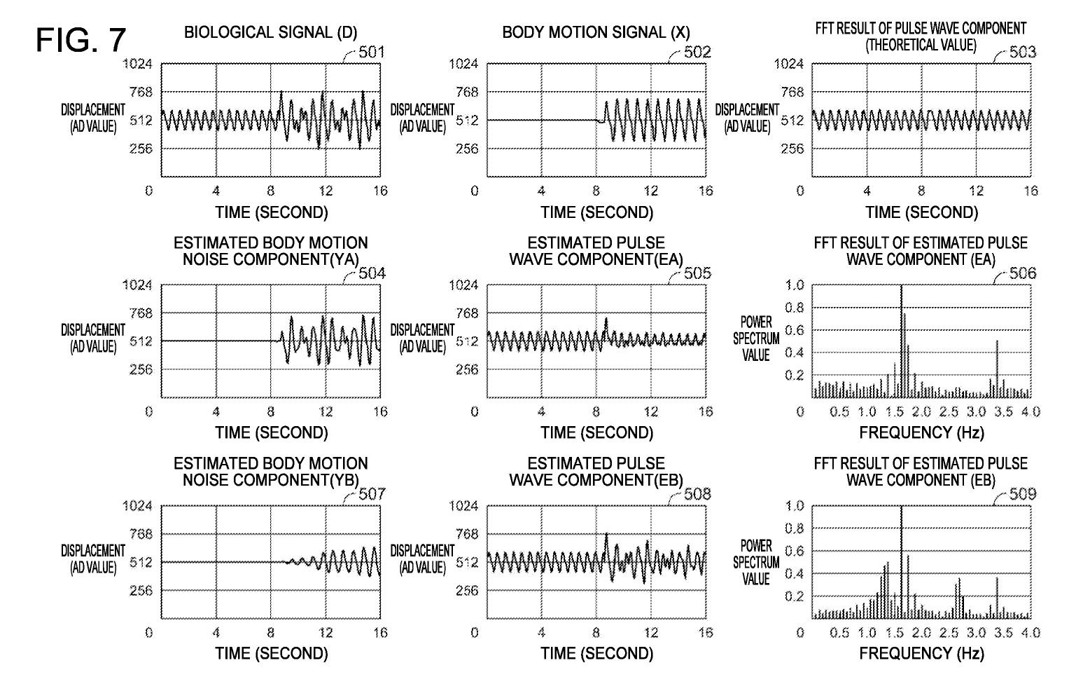

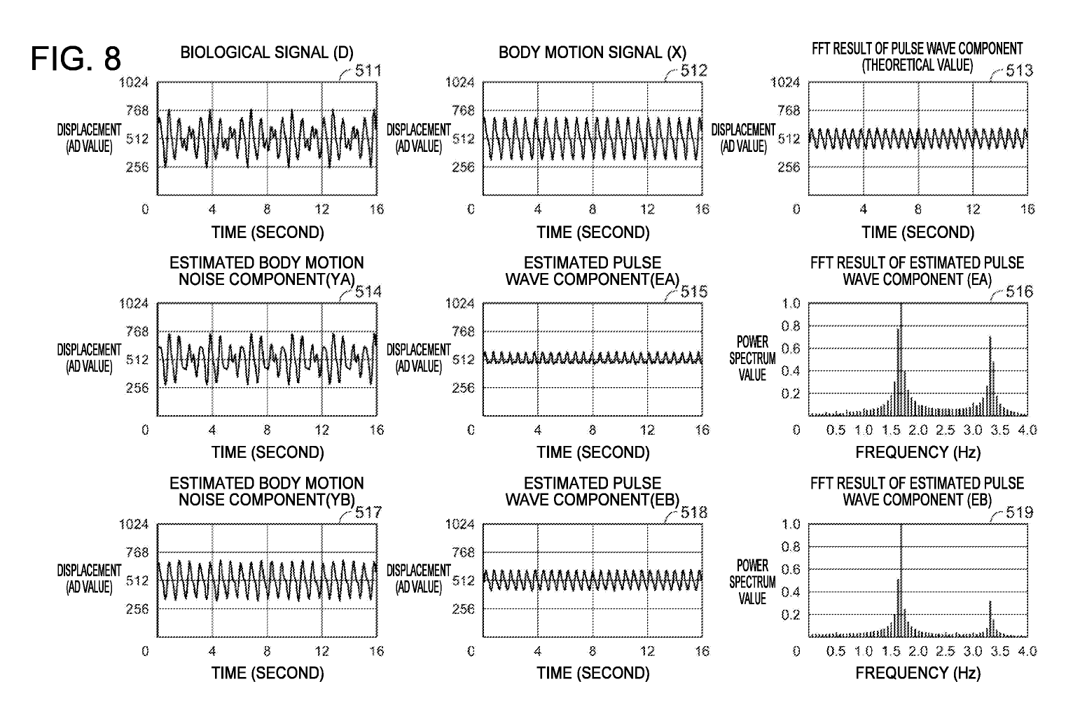

FIG. 7 and FIG. 8 are graphs showing application examples of the adaptive filter. FIG. 7 and FIG. 8 are simulation data derived on the basis of an experiment measured during exercise (running) of the subject. FIG. 7 is a signal data obtained by assuming an exercise start time of the subject and a processing result of the signal data. FIG. 8 is signal data obtained by assuming that running is steadily performed at the same pitch after the elapse of an exercise time of the subject. A value of the step size of the adaptive filter A 110 is set to a value larger than a value of the step sizeμ of the adaptive filter B 120.

A biological signal 501 is the biological signal D detected by the biological-signal detecting unit. The ordinate indicates an AD value representing displacement of a waveform of the biological signal D and the abscissa indicates a measurement time (second). In the following explanation, a graph showing waveforms of a signal and a signal component has the same coordinate axes.

A body motion signal 502 is the body motion signal X detected by the body-motion-signal detecting unit and is acceleration data in the X-axial direction. In the biological signal 501, the displacement of the waveform cyclically appears in a range of approximately 420 to 600 in a period of 1 to approximately 8 seconds. The displacement of the waveform appears as a waveform with large amplitude in a range of approximately 250 to 780 in a period of approximately 8 to 16 seconds. In the waveform of the body motion signal 502, a waveform up to a point in time of 0 to approximately 8 seconds is a substantially straight line. The displacement of the waveform cyclically appears in a range of approximately 320 to 700 in a period of 8 to approximately 16 seconds. A peak (approximately 700) appears ten times in 8 seconds. These waveforms are obtained by simulating sensor signals before and after a simple exercise start.

A pulse wave component (a theoretical value) 503 is a theoretical value calculated by simulating a pulse wave component not including noise.

All of an estimated body motion noise component 504, an estimated pulse wave component 505, and an estimated pulse wave component 506 are results obtained by applying the adaptive filter A 110. An estimated body motion noise component 507, an estimated pulse wave component 508, and an estimated pulse wave component 509 are results obtained by applying the adaptive filter B 120. Graphs of the estimated pulse wave component 506 and the estimated pulse wave component 509 are graphs representing a power spectrum value at each frequency by subjecting an estimated pulse wave component to FFT processing. The ordinate indicates intensity of a power spectrum value and the abscissa indicates a frequency (Hz).

The estimated body motion noise component 504 is an estimated body motion noise component calculated from the body motion signal 502 by applying the adaptive filter A 110. The estimated body motion noise component 507 is an estimated body motion noise component calculated from the body motion signal 502 by applying the adaptive filter B 120. In the estimated body motion noise component 504, the displacement of the waveform is in a range of approximately 350 to 740 in a period of approximately 8 to 16 seconds. A change is seen in the displacement of the waveform compared with the body motion signal 502. In the estimated body motion noise component 507, the displacement of the waveform starts to appear from approximately 9 seconds. The displacement of the waveform is gradually amplified. The displacement of approximately 420 to 590 appears in the vicinity of 16 seconds.

The estimated pulse wave component 505 is a waveform obtained by subtracting the estimated body motion noise component 504 from the biological signal 501. The estimated pulse wave component 508 is a waveform obtained by subtracting the estimated body motion noise component 507 from the biological signal 501. In the estimated pulse wave component 508, the displacement of the waveform changed to a range of approximately 320 to 770 appears in a period of approximately 8 to 16 seconds. In the estimated pulse wave component 505, the displacement of the waveform cyclically appears in a narrow range of approximately 460 to 580 in a period of approximately 8 to 16 seconds. The waveform is stable compared with the estimated pulse wave component 508.

The estimated pulse wave component 506 represents a power spectrum value of the estimated pulse wave component 505. The estimated pulse wave component 509 represents a power spectrum value of the estimated pulse wave component 508. In both of the estimated pulse wave component 506 and the estimated pulse wave component 509, a strongest base line is a frequency of 1.625 Hz. The frequency of 1.625 Hz is considered to be a pulse wave component because the frequency indicates a strongest base line (not shown in the figure) in a frequency component of the pulse wave component (the theoretical value) 503. In the estimated pulse wave component 509, relatively strong base lines remain in the vicinity of a frequency of approximately 1.3 Hz and in the vicinity of a frequency of approximately 2.7 Hz. These frequencies indicate a strong base line (not shown in the figure) in a frequency component of the body motion signal 502 as well. The frequencies are remaining of the body motion noise component. In the estimated pulse wave component 506, strong base lines do not appear in the vicinity of the frequency of approximately 1.3 Hz and in the vicinity of the frequency of approximately 2.7 Hz. Therefore, it is seen that remaining of the body motion noise component is small.

As explained above, at the exercise start time of the subject, it can be confirmed that the adaptive filter A 110 that calculates the estimated pulse wave component 506 can attenuate the body noise component more than the adaptive filter B 120.

Application examples of the adaptive filter to signal data assumed after the elapse of an exercise time of the subject are explained with reference to FIG. 8. The arrangement and the type of graphs are the same as those in FIG. 7. A biological signal 511 is the detected biological signal D, a body motion signal 512 is the detected body motion signal X, and a pulse wave component (a theoretical value) 513 is a theoretical value calculated by simulating a pulse wave component. All of an estimated body motion noise component 514, an estimated pulse wave component 515, and an estimated pulse wave component 516 are results obtained by applying the adaptive filter A 110. An estimated body motion noise component 517, an estimated pulse wave component 518, and an estimated pulse wave component 519 are results obtained by applying the adaptive filter B 120. A measurement time is 16 seconds. Measurement is performed while substantially maintaining an exercise state for 0 to 16 seconds.

In the biological signal 511, as displacement of a waveform, a waveform fluctuating in a range of approximately 250 to 780 appears. In the body motion signal 512, the displacement of the waveform cyclically appears in a range of approximately 320 to 700. A peak (approximately 700) appears twenty-one times in 16 seconds. This waveform is signal data obtained by simulating a situation in which running is continued at a pace of 78 times/minute of an arm swinging interval in 16 seconds and a pace of approximately 156 (steps/minute) as a pitch.

In the estimated body motion noise component 514 calculated by applying the adaptive filter A 110, the displacement of the waveform changed to a range of approximately 280 to 750 appears. In the estimated body motion noise component 517 calculated by applying the adaptive filter B 120, the displacement of the waveform cyclically appears in a range of approximately 350 to 690. The number of times of a peak is twenty-one same as the number of times of the peak of the estimated body motion signal 512. The shape of the waveform is similar to the shape of the waveform of the estimated body motion signal 512. In the estimated pulse wave component 515, the displacement of the waveform cyclically appears in a relatively narrow range of approximately 480 to 570. In the estimated pulse wave component 518, the displacement of the waveform cyclically appears in a range of approximately 420 to 600. When the waveforms of the estimated pulse wave component 515 and the estimated pulse wave component 518 are compared with the pulse wave component (the theoretical value) 513, the shape of the waveform of the estimated pulse wave component 518 is similar to the shape of the waveform of the pulse wave component (the theoretical value) 513.

In power spectrum values of the estimated pulse wave component 516 and the estimated pulse wave component 519, power spectrum distribution shapes of both of the estimated pulse wave components 516 and 519 are similar. The frequencies of strongest base lines of both of the estimated pulse wave components 516 and 519 are approximately 1.687 Hz. However, a ratio of a side lobe to a main lobe is smaller in the estimated pulse wave component 519 than in the estimated pulse wave component 516. That is, a noise component is reduced more in the estimated pulse wave component 519 than in the estimated pulse wave component 516.

As explained above, after the elapse of the exercise time of the subject, it can be confirmed that the body motion noise component is reduced more in the adaptive filter B 120 that calculates the estimated pulse wave component 519 than in the adaptive filter A 110.

In this way, for the body motion signal in which the cyclicity of exercise suddenly changes as at the exercise start time, the adaptive filter having the learning characteristics with the increased value of the step size μ extracts the estimated pulse wave component with the body motion noise component sufficiently attenuated. For the body motion signal in which the cyclicity of exercise is stable as at the exercise continuation time, the adaptive filter having the learning characteristics with the reduced value of the step size μ extracts the estimated pulse wave component with the body motion noise component sufficiently attenuated.

By setting different values as the step size μ of the learning characteristics, it is possible to cope with a change in cyclicity of exercise and stable various situations. However, the estimated pulse wave component with the body motion noise component sufficiently attenuated according to various situations cannot be estimated with a single learning characteristic. Therefore, the adaptive filter includes a plurality of adaptive filters having different learning characteristics. It is determined using a correlation coefficient which signal is selected out of output signals (estimated pulse wave components and estimated body motion noise components) from the respective adaptive filters. The correlation coefficient is an index indicating a degree of a correlation between the body motion signal X and output signals from the adaptive filters.

### (Determination by the Correlation Coefficient)

Details of the correlation coefficient are explained. The correlation coefficient is a coefficient calculated by the correlation-information calculating unit 130 shown in FIG. 5. The correlation coefficient CA is calculated on the basis of an output signal of the adaptive filter A 110. The correlation coefficient CB is calculated on the basis of an output signal of the adaptive filter B 120.

The respective estimated pulse wave components EA and EB calculated by the adaptive filter A 110 and the adaptive filter B 120 are signal components with the noise components related to the body motion signal X attenuated. Therefore, as the correlation degree with the body motion signal X is weaker, the body motion noise component is more sufficiently attenuated. The correlation degree is determined using the correlation coefficient. The correlation coefficient is calculated by Formulas (5) to (8). In Formulas (5) to (8), a data sequence 1 is represented as d1(i) and a data sequence 2 is represented as d2(i) and a correlation coefficient C is calculated. In the formulas, d1m represents an average of the data sequence 1 and d2m represents an average of the data sequence 2, i is a natural number and is a numerical value from 1 to the number of data n, and Vx, Vy, and Vxy represents parameters.

Vx=Σ[d1(i)−d1m]² i=1 to n Formula (5)

Vy=Σ[d2(i)−d2m]² i=1 to n Formula (6)

Vxy=Σ{[d1(i)−d1m]·[d2(i)−d2m]} i=1 to n Formula (7)

C=Vxy/(√Vx·⇄Vy) Formula (8)

Data sequences of the estimated pulse wave component EA and the body motion signal X calculated by the adaptive filter A 110 are applied to Formulas (5) to (8) as the data sequence 1 and the data sequence 2 and the correlation coefficient is calculated. The calculated correlation coefficient is represented as CA. A correlation function calculated by applying data sequences of the estimated pulse wave component EB and the body motion signal X calculated by the adoptive filter B 120 to Formulas (5) to (8) is represented as CB. The correlation coefficient has a range of −1 to +1. The correlation degree is weaker as the correlation coefficient is closer to 0. The correlation degree is stronger as the correlation coefficient is closer to +1 and −1. Therefore, the magnitudes (the absolute values) of the correlation coefficient CA and the correlation coefficient CB are compared and a smaller estimated pulse wave component is selected. The body motion noise component is sufficiently attenuated in the selected estimated pulse wave component compared with the estimated pulse wave component not selected. The learning characteristics of the adaptive filter that outputs the selected estimated pulse wave component is more suitable for the body motion signal X and attenuates the body motion noise component more than the learning characteristics of the adaptive filter that outputs the estimated pulse wave component not selected.

In this way, in any situation of a change in cyclicity of exercise, a stable state, or the like, it is possible to select the estimated pulse wave component suitable for the body motion signal X and with noise attenuated using the correlation coefficient out of the estimated pulse wave components output from the provided plurality of adaptive filters.

### (Control Program of the Pulsimeter)

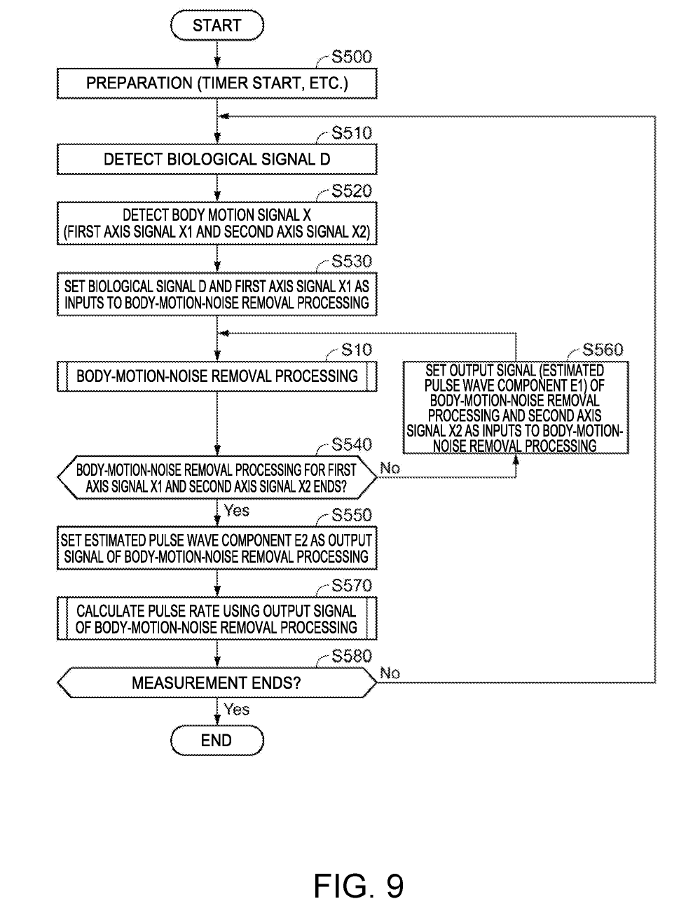

FIG. 9 is a flowchart for explaining a flow of processing of the control program of the pulsimeter. The flow of the processing is explained below mainly with reference to FIG. 9 and with reference to FIG. 4 to FIG. 6 as well. Note that the flow explained below is equivalent to the biological-information processing method and is executed by the control unit 200 controlling the units including the storing unit 260 on the basis of the control program 261 stored in the storing unit 260. According to the execution of the control program 261, functions of the functional units including the pulse wave sensor 10, the acceleration sensor 20, the pulse-wave-AD converting unit 30, the acceleration-AD converting unit 40, the pulse-rate calculating unit 60, and the body-motion-noise removing unit 100 are realized.

In step S500, preparation for biological signal and body motion signal detection by the pulse wave sensor 10 and the acceleration sensor 20 is performed. Specifically, first, a time is set using a real time clock of the clocking unit 250. The timer sets at least sampling cycles of the pulse wave sensor 10, the acceleration sensor 20, the pulse-wave-AD converting unit 30, and the acceleration-AD converting unit 40. The timer sets a predetermined period for calculating a pulse rate. When time such as 1 to 6 seconds is set, the pulse rate is calculated once in 1 to 6 seconds.

In step S510, the biological signal D is detected. Specifically, a biological signal is detected by the pulse wave sensor 10 for a predetermined period. The biological signal of an analog signal is converted into the biological signal D of a digital signal by the pulse-wave-AD converting unit 30. Note that step S510 is equivalent to the biological-signal detecting step.

In step S520, a body motion signal is detected. Specifically, a body motion signal is detected by the acceleration sensor 20 for a predetermined period. The body motion signal of an analog signal is converted into the body motion signal X of a digital signal by the acceleration-AD converting unit 40. As the body motion signal X, acceleration signals in the X-axial direction and the Y-axial direction are detected out of acceleration signals in the X-axis, Y-axis, and Z-axial directions detected by the acceleration sensor 20. The X-axial direction is represented as a first axial direction X1 and the Y-axial direction is represented as a second axial direction X2. Note that step S520 is equivalent to the body-motion-signal detecting step.

In step S530, the biological signal D and the first axis signal X1 serving as the body motion signal are set as inputs to body-motion-noise removal processing S10. Specifically, the detected biological signal D is selected and the first axis signal X1 is selected from the body motion signal X. The biological signal D and the first axis signal X1 are set as input signals to the body-motion-noise removal processing S10 to be performed next. In the body-motion-noise removal processing S10, an estimated body motion noise component calculated from the biological signal D on the basis of the first axis signal X1 is subjected to attenuation processing.

Step S10 is a subroutine program for attenuating a body motion noise component. Processing for attenuating the body motion noise component from the biological signal D using the body motion signal X is performed and an estimated pulse wave component is output. The sub-routine program is a program for realizing a function of the body-motion-noise removing unit 100 functioning as a functional unit and includes the functions of the adaptive filter A 110, the adaptive filter B 120, the correlation-information calculating unit 130, and the selecting unit 140. Details of the subroutine program are explained below.

In step S540, it is checked whether the body-motion-noise removal processing S10 using body motion signals of both of the first axis signal X1 and the second axis signal X2 is performed. When processing of both of the first axis signal X1 and the second axis signal X2 ends (Yes), the processing proceeds to step S550. When only the first axis signal X1 is processed (No), the processing proceeds to step S560 and proceeds to processing for the second axis signal X2.

In step S550, the estimated pulse wave component E2 output from the body-motion-noise removal processing S10 is set as an output signal. Specifically, the estimated pulse wave component E2 with a noise component sufficiently attenuated on the basis of the first axis signal X1 and the second axis signal X2 superimposed on the biological signal D is output by the body-motion-noise removal processing S10.

In step S560, the estimated pulse wave component E1 and the second axis signal X2 serving as a body motion signal are set as inputs to the body-motion-noise removal processing S10. Specifically, the estimated pulse wave component E1 with a noise component related to the first axis signal X1 attenuated from the biological signal D and the second axis signal X2 are selected and set as input signals to the body-motion-noise removal processing S10 to be performed next. In the body-motion-noise removal processing S10, an estimated body motion noise component calculated from the estimated pulse wave component E1 on the basis of the second axis signal X2 is subjected to attenuation processing.

Step S570 is a subroutine program for calculating a pulse rate. The pulse rate is calculated using the estimated pulse wave component E2, which is an output signal of the body-motion-noise removal processing S10. Specifically, FFT processing is performed to specify a frequency component equivalent to the pulse rate. The pulse rate is calculated from the specified frequency component. The subroutine program is a program for realizing a function of the pulse-rate calculating unit 60 functioning as a functional unit. In a process of the FFT processing, if noise is less in the estimated pulse wave component E2, a frequency assuming a pulse is more easily specified.

In step S580, it is determined whether the pulse measurement is ended. Specifically, in steps S500 to S570 and step S10, when the operation button 5 (FIG. 1) indicating a measurement end is depressed by the subject (Yes), processing including body-motion-noise removal processing and pulse rate measurement processing of the control program 261 is ended. When the operation button 5 is not depressed (No), the processing proceeds to step S20. Processing including the body-motion-noise removal processing and the pulse rate measurement processing is performed from a biological signal detected in the next predetermined period.

### (Subroutine Program of the Body-Motion-Noise Removal Processing)

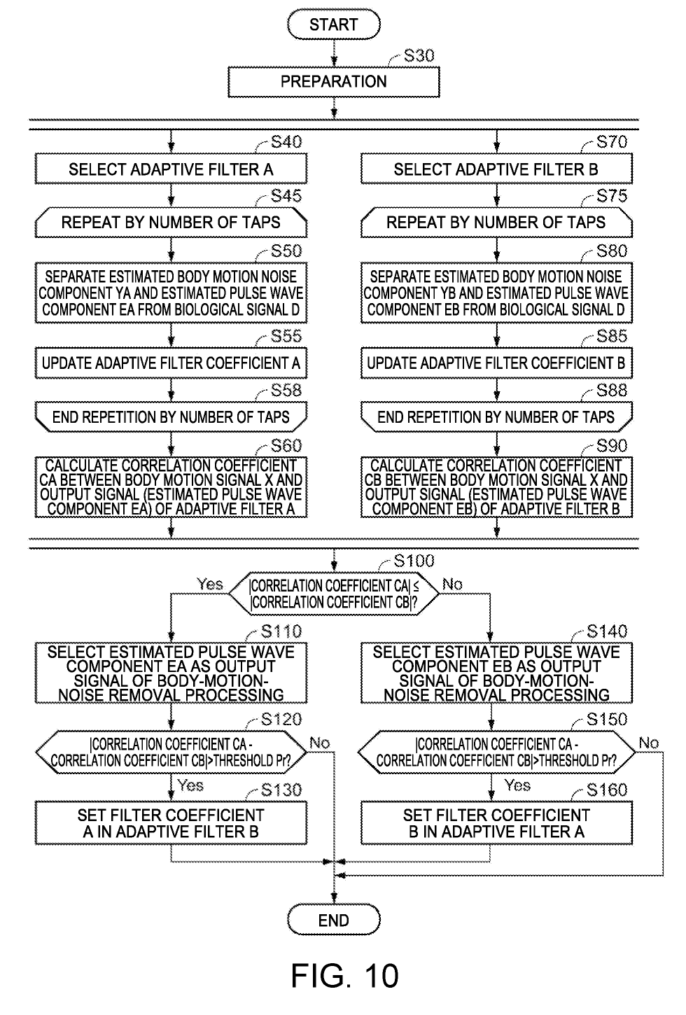

FIG. 10 is a flowchart for explaining a flow of the body-motion-noise removal processing. The flow of the body-motion-noise removal processing is explained below mainly with reference to FIG. 10 and with reference to FIG. 4 to FIG. 6 and FIG. 9 as well. Note that the flow explained below is equivalent to the body-motion-noise removal processing step and is executed by the control unit 200 controlling the units including the storing unit 260 using the flow as a subroutine program, which is a part of the control program 261 stored in the storing unit 260. The flow is a subroutine program invoked from step S60 (the body-motion-noise removal processing) in the flow of the control program 261.

In step S30, preparation for subroutine program execution of the body-motion-noise removal processing is performed. For example, initialization of variables and a storage region used in the subroutine program is performed.

Steps S40 to S60 and steps S70 to S90 are processing groups subjected to parallel processing. The respective processing groups are started after the execution of step S30 ends. When the respective processing groups end, step S100 is started. The parallel processing may be realized by adopting a pseudo multitask structure by the control program 261 or may be realized by mounting a plurality of MPUs or DSPs on the control unit 200 and causing the MPUs or the DSPs to share processing. Note that steps S40 to S58 and steps S70 to S88 are equivalent to the filter step. Steps S60 and step S90 are equivalent to the correlation-information calculating step.

In step S40, an adaptive filter A is selected as a filter used for the signal extraction processing. Specifically, a filter coefficient A including a step size A having learning characteristics of the adaptive filter A is set in the filter-coefficient setting unit 153.

In step S45, preprocessing of repetition processing for a predetermined period, for example, for the number of taps of the filter is performed. Specifically, for example, processing in steps S45 to S58 is repeated until output signals of a predetermined number of samples, for example, for four seconds are obtained. The number of taps of the filter coincides with the number of adaptive filter coefficients A.

In step S50, an estimated biological noise component YA and an estimated pulse wave component EA are separated from the biological signal D. Specifically, the estimated body motion noise component YA is calculated using the body motion signal X and the filter coefficient A. A difference of the estimated body motion noise component YA is calculated from the biological signal D and the estimated pulse wave component EA is calculated.

In step S55, the filter coefficient A is updated. Specifically, the step size A is calculated using the body motion signal X. The filter coefficient A is updated using the step size A, the estimated body motion noise component YA, and the calculated estimated pulse wave component EA. The filter coefficient A is calculated by the number of tap sizes.

In step S58, the repetition for the predetermined period, for example, for the number of taps of the filter is ended. The processing proceeds to step S45 until the processing by the number of taps of the filter is repeated in steps S45 to S58. When the processing by the number of taps ends, the processing proceeds to the next step S60.

In step S60, the correlation coefficient CA between the body motion signal X and the estimated pulse wave component EA is calculated. Specifically, in the correlation-information calculating unit 130, the body motion signal X and the estimated pulse wave component EA output from the adaptive filter A are input. The body motion signal X and the estimated pulse wave component EA are applied to Formula (5) to Formula (8) to calculate the correlation coefficient CA. Note that the estimated pulse wave component EA output from the adaptive filter A is equivalent to the output signal from the filter unit.

In steps S70 to S90, the processing is performed using an adaptive filter B in a procedure same as steps S40 to S60. In the adaptive filter B, a filter coefficient B including a step size B different from the learning characteristics of the adaptive filter A is set in the filter-coefficient setting unit 153 of the adaptive filter B. In a process of setting the filter coefficient B, various data including the estimated pulse wave component EB, the estimated body motion noise component YB, the filter coefficient B, and the correlation coefficient CB are generated.

In step S100, the absolute value of the correlation coefficient CA and the absolute value of the correlation coefficient CB are compared. Specifically, the correlation coefficients calculated in steps S60 and S90 are correlation coefficients between the estimated pulse wave components and the body motion signal. Therefore, as the correlation of the estimated pulse wave component with the body motion signal is weaker, the body motion noise component can be more attenuated. Therefore, as the absolute value of the correlation coefficient is smaller, a pulse wave component with less body motion noise can be calculated. If the absolute value of the correlation coefficient CA is equal to or smaller than the absolute value of the correlation coefficient CB (Yes), it is determined that the correlation of the estimated pulse wave component EA with the body motion signal X is low. The processing proceeds to step S110. If the absolute value of the correlation coefficient CA is larger than the absolute value of the correlation coefficient CB (No), it is determined that the correlation of the estimated pulse wave component EB with the body motion signal X is low. The processing proceeds to step S140.

In step S110, the estimated pulse wave component EA is selected as an output signal of the body-motion-noise removal processing. Specifically, since the correlation of the estimated pulse wave component EA with the body motion signal X is low in step S100, the estimated pulse wave component EA is a signal component which involves the attenuated noise having higher correlation with the body motion signal X. That is, in the estimated pulse wave component EA calculated by the adaptive filter A, noise component is less than noise component in the estimated pulse wave component EB calculated by the adaptive filter B. The selecting unit 140 selects the estimated pulse wave component EA, which is the output signal of the adaptive filter A, as an output signal of the body-motion-noise removal processing. The selected estimated pulse wave component EA is input to a subroutine for calculating a pulse rate in step S570. The pulse rate is calculated on the basis of the estimated pulse wave component EA.

In step S120, it is determined whether the absolute value of a difference between the correlation coefficient CA and the correlation coefficient CB is larger than a predetermined threshold Pr. Specifically, when the difference between the correlation coefficient CA and the correlation coefficient CB is larger (larger than the predetermined threshold Pr), a correlation degree diverges. Therefore, the accuracy of the adaptive filter B is not improved. The filter coefficient B needs to be adjusted. Therefore, if the correlation degree is larger than the predetermined value β r (Yes), it is determined that the adjustment of the adaptive filter B is necessary. The processing proceeds to step S130. If the correlation degree is smaller than the predetermined threshold Pr, the processing proceeds to step S170.

In step S130, the filter coefficient CA is set in the adaptive filter B. Specifically, in the filter-coefficient-setting unit 153, a value of the filter coefficient CB set in step S70 is set to a value of the latest filter coefficient CA updated in step S55. Specifically, the value of the filter coefficient CA is substituted in h(l) to h(L) used first as a setting value of h(k) of Formula (3) for calculating a filter coefficient of the adaptive filter B.

In step S140 to step S160, the processing is performed targeting the adaptive filter B in a procedure same as steps S110 to S130. In a process of the processing, the selecting unit 140 selects the estimated pulse wave component EB and sets the estimated pulse wave component EB as an output signal of the body-motion-noise removal processing. When the difference between the absolute value of the correlation coefficient CB and the absolute value of the correlation coefficient CA is larger than the predetermined threshold Pr, the filter coefficient CB is set as a value of the adaptive filter A. Note that steps S100 to S160 are equivalent to the selecting step. Specifically, the filter coefficient A including the step size A having the learning characteristics of the adaptive filter A is set in the filter-coefficient setting unit 153. Note that, in the above explanation, the difference between the correlation coefficients and the threshold are compared to control the setting of the filter coefficients. However, for example, a ratio of the correlation coefficients of the filters, for example, CA/CB may be calculated and compared with a threshold.

As explained above, the estimated pulse wave component EA and the estimated pulse wave component EB respectively calculated using the adaptive filter A 110 and the adaptive filter B 120 having the different learning characteristics are compared with the body motion signal X. The estimated pulse wave component having a lower correlation is selected as a signal that should be output. With an estimated pulse wave component calculated using the one adaptive filter in the past, body motion noise temporarily cannot be sufficiently attenuated depending on a situation of a body motion signal. However, it is possible to extract a pulse wave component with less noise component from extraction results of a plurality of adaptive filters in every predetermined period.

If the difference between the correlation coefficients is larger than the predetermined threshold, the filter coefficient calculated by the adaptive filter that calculates the selected estimated pulse wave component is set as the filter coefficient of the adaptive filter that calculates the estimated pulse wave component not selected.

Consequently, it is possible to improve the performance of the adaptive filter that calculates the estimated pulse wave component not selected. That is, both the adaptive filters have even performance from a point in time when the filter coefficients are set in the adaptive filters. Thereafter, it is possible to perform adaptive processing (learning processing) based on the respective learning characteristics. Therefore, since the filter performances of the respective plurality of filters having the different learning characteristics are improved, features of the learning characteristics are directly reflected. It is possible to extract the estimated pulse wave component with the estimated body motion noise more precisely attenuated.

Note that, in this embodiment, the adaptive filter includes the two kinds of adaptive filters. However, the adaptive filter may include adaptive filters having three or more kinds of a plurality of different learning characteristics.

### (Effects)

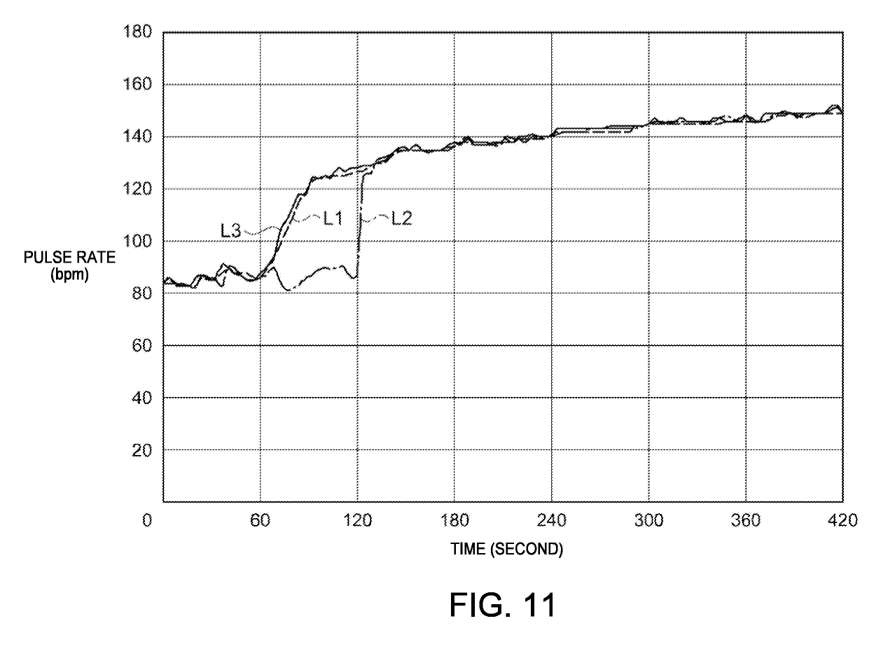

An example of effects of the pulsimeter 1 applied with this embodiment is explained with reference to FIG. 11.

FIG. 11 is a graph representing an example of a calculated pulse rate. The abscissa of the graph in FIG. 11 indicates an elapsed time (second) and the ordinate indicates a pulse rate (bpm) (beats per minute). Graphs are a pulse rate L1 (a dotted line) indicated by the pulsimeter 1 including the adaptive filter A 110 and the adaptive filter B 120 in this embodiment, a pulse rate L2 (an alternate long and short dash line) indicated by the pulsimeter configured by one adaptive filter in the past, and a heart rate L3 (a solid line) of the subject. The heart rate L3 is a heart rate measured by a Holter electrocardiograph or the like. Note that the pulse rate is a numerical value calculated by the pulse-rate calculating unit 60 (FIG. 4) from an estimated pulse wave component selected by the body-motion-noise removing unit 100 (FIG. 4).

The heart rate L3 of the subject is stable at approximately 85 to 90 bpm in a period of 0 to approximately 60 seconds. The pulse rate L1 and the pulse rate L2 indicate values substantially close to the heart rate L3. The subject starts exercise at approximately 60 seconds. After the exercise start, the heart rate L3 suddenly rises to a pulse rate of 90 bpm to 130 bpm while drawing a curve of a mountain shape in a period of approximately 60 to 120 seconds. The pulse rate L2 indicates a pulse rate of approximately 85 to 90 bpm in the period of approximately 60 to 120 seconds and cannot follow the actual heart rate L3. This is a result of, in the adaptive filter mounted on the pulsimeter in the past, inability to sufficiently attenuate a body motion noise component due to a change in an exercise state and discriminate a base line of a pulse wave component.

The pulse rate L1 transitions generally the same in the vicinity of the curve of the actual heart rate L3 in the period of approximately 60 to 120 seconds. Further, after approximately 120 seconds, the pulse rate gradually increases from approximately 130 bpm to approximately 150 bpm according to an exercise load and indicates generally the same transition as the heart rate L3. The pulse rate L1 is a result of selecting an estimated pulse wave component by the adaptive filter A 110 in the period of approximately 60 to 120 seconds, selecting an estimated pulse wave component by the adaptive filter B 120 after approximately 120 seconds, and calculating a pulse rate.

The pulse rate L1 in this embodiment is present in the vicinity of the curve of the transition of the actual heart rate L3 throughout the measurement. A numerical value close to the heart rate of the subject can be calculated. That is, it is seen that an estimated pulse wave component used for the calculation of the pulse rate has a base line strongly showing a frequency component of a pulse wave in a process of FFT processing and noise component is less.

As a result of applying this embodiment to the actual pulsimeter 1 and verifying the pulse rates, an estimated pulse wave component with a body motion noise component, which fluctuates according to a change in an exercise state of the subject, sufficiently attenuated. The pulse rates close to the actual heart rate can be calculated.

### Second Embodiment

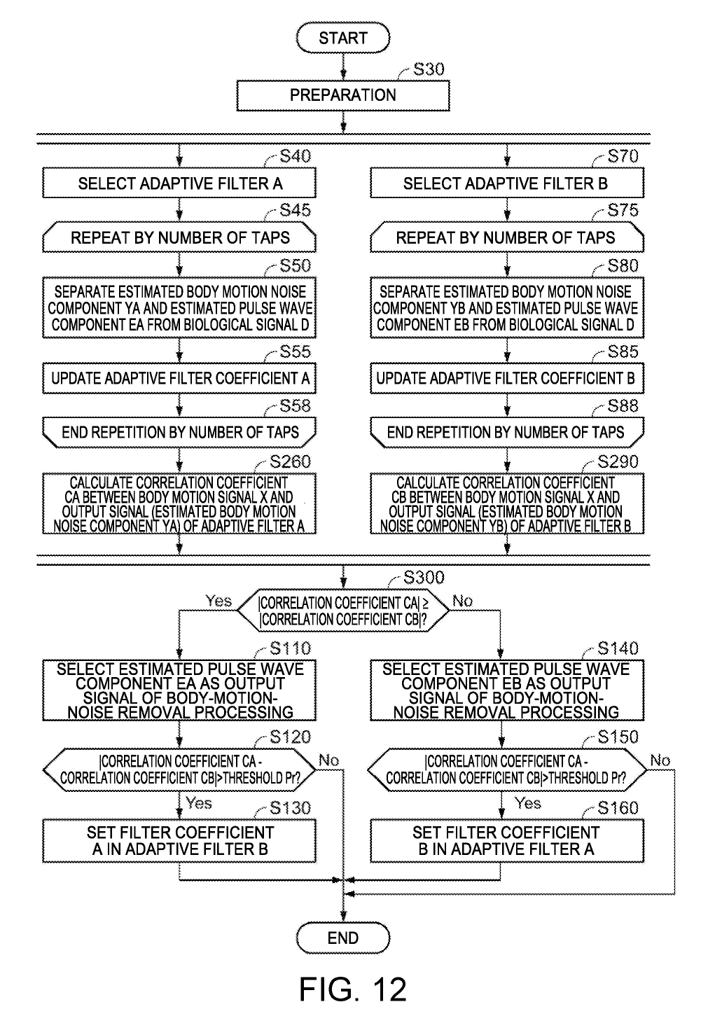

A second embodiment is explained mainly with reference to FIG. 12 and with reference to the figures as appropriate.

FIG. 12 is a flowchart for explaining a flow of body-motion-noise-component removal processing in the second embodiment. In this embodiment, a part of the flow (FIG. 10) showing the flow of the body-motion-noise-component removal processing in the first embodiment is different. Note that the flow explained below is equivalent to the biological-information processing method and executed by the control unit 200 controlling the units including the storing unit 260 on the basis of the control program 261 stored in the storing unit 260.

In the first embodiment, in step S60 and step S90 serving as the correlation-coefficient calculating step, the correlation coefficient between the body motion signal X and the estimated pulse wave component EA or the estimated pulse wave component EB is calculated. This embodiment is different in that, as step S260 and step S290, a correlation coefficient between the estimated body motion noise component YA or the estimated body motion noise component YB and the body motion signal X is calculated. This embodiment is different in that the determination “if the absolute value of the correlation coefficient CA is equal to or smaller than the absolute value of the correlation coefficient CB” in step S100 serving as the processing of a part of the selecting step in the first embodiment is changed to determination “if the absolute value of the correlation coefficient CA is equal to or larger than the absolute value of the correlation coefficient CB” in step S300 in this embodiment.

In step S260, the correlation coefficient CA between the estimated body motion noise component YA and the body motion signal X is calculated. Specifically, the body motion signal X and the estimated body motion noise component YA, which is an output of the adaptive filter A 110, are input to the correlation-information calculating unit 130. The body motion signal X and the estimated body motion noise component YA are applied to Formula (5) to Formula (8) to calculate the correlation coefficient CA.

In step S290, the correlation coefficient CB between the estimated body motion noise component YB and the body motion signal X is calculated. Specifically, the body motion signal X and the estimated body motion noise component YB, which is an output of the adaptive filter B 120, are input to the correlation-information calculating unit 130. The body motion signal X and the estimated body motion noise component YB are applied to Formula (5) to Formula (8) to calculate the correlation coefficient CB.

In step S300, the absolute value of the correlation coefficient CA and the absolute value of the correlation coefficient CB are compared. Specifically, the correlation coefficients calculated in step S260 and S290 are correlation coefficients between the estimated body motion noise components and the body motion signal. Therefore, as the correlation of the estimated body motion noise component with the body motion signal is stronger, the body motion noise component can be calculated following the body motion noise more. Therefore, as the absolute value of the correlation coefficient is larger, the estimated body motion noise component following the body motion noise more can be calculated. The remaining of the body motion noise is less in the extracted estimated pulse wave component. Therefore, if the absolute value of the correlation coefficient CA is equal to or larger than the absolute value of the correlation coefficient CB (Yes), since the body motion noise is less in the estimated pulse wave component EA, the processing proceeds to step S110. The estimated pulse wave component EA is selected as an output single. If the absolute value of the correlation coefficient CA is equal to or smaller than the absolute value of the correlation coefficient CB (No), since body motion noise is calculated less in the estimated pulse wave component EB, the processing proceeds to step S140. The estimated pulse wave component EB is selected as an output signal.

As explained above, in this embodiment, effects same as the effects in the first embodiment can be obtained by calculating the correlation coefficients for the body motion signal and the estimated body motion noise components and selecting the estimated pulse wave component.

This embodiment may be used in combination with the first embodiment. For example, the adaptive filter includes three or more adaptive filters, the determination of the correlation coefficient by the first embodiment is applied in order to exclude one adaptive filter from the three adaptive filters, and the determination by the correlation function in this embodiment is applied to the remaining two adaptive filters. Consequently, there is possibility that it is possible to extract an estimated pulse wave component with a noise component more attenuated.

Note that the present invention is not limited to the embodiments explained above. It is possible to apply various changes and improvements to the embodiments. Modifications are explained below.

### (Modification 1)

In the embodiments explained above, the configuration is adopted in which the acceleration sensor 20 is provided in the body-motion-signal detecting unit. However, the present invention is not limited to this configuration. A configuration may be adopted in which a contact pressure sensor is provided and a detected contact pressure displacement amount signal is included in a body motion signal. Specifically, the contact pressure sensor is a sensor that is, in a state in which the pulsimeter 1 is worn on the arm, disposed adjacent to an arm contact surface side of the pulse wave sensor 10 and measures a displacement amount of physical pressing that occurs between the pulse wave sensor 10 and the arm. Clenching and opening actions of the hand, a shift of the arm wearing state of the pulsimeter 1, or the like is mainly detected as the contact pressure displacement amount signal.

Extraction processing for an estimated pulse wave component is performed on the basis of such a contact pressure displacement amount signal. Consequently, it is possible to attenuate a body motion noise component such as the clenching and opening actions of the hand, the shift of the arm wearing state, or the like superimposed on a biological signal.

### (Modification 2)

In the embodiments and the modification explained above, the calculation formula for the filter coefficient of the adaptive filter is Formula (3). However, the filter coefficient may be updated using Formula (9).

h(k)=h(k)+μ·e(i)/[p(i−k)/Σp²(i−k)] k=1 to L Formula (9)

In Formula (9), p(i−k) is a coefficient calculated from a delay signal x(i−k) of a body motion signal on the basis of an affine projection method and is calculated by Formula (10).

p(i−k)=x(i−k)+[{Σx[(i−k)·x(i−k−l)]}/[Σx²(i−k−l)]]·x(i−k) k=1 to L Formula (10)

Further, in the update of the filter coefficient, not only the embodiments and the modification but also another formula involving different arithmetic expression may be used. For example, an algorithm such as LMS or nLMS may be used. An adaptive filter having at least two or more kinds of adaptive algorithms and learning characteristics only has to be used. The number of taps (a value of L) of the filter coefficient h(k) only has to be the same. However, the number of taps of the filter coefficient does not have to be the same number of taps when processing for setting the filter coefficient in another adaptive filter is not included. Consequently, a calculation method for an estimated body motion noise component is diversified. The possibility of calculating an estimated body motion noise component that can follow a fluctuating body motion signal expands. As a result, it is possible to extract an estimated pulse wave component with minimized noise.

### (Modification 3)

In the embodiments and the modifications explained above, the order of applying the acceleration signals in the X-axis, Y-axis, and Z-axial directions to the adaptive filter is not specified. However, the adaptive filter may be applied in order from the axis with the largest motion in the three axes of the X axis, the Y axis, and the Z axis. Specifically, first, in the body-motion-signal detecting unit, sums of amounts of changes of acceleration signals in the axial directions are calculated and stored. A body motion signal input to the body-motion-noise removing unit 100 is applied in order from an acceleration signal in the axial direction with the largest sum of the amounts of changes. Body motion noise in the axial directions is removed from a biological signal. The sums of the amounts of changes of the acceleration signals are values representing motions of the subject. Therefore, a body motion noise component having the largest influence superimposed on the biological signal can be removed in order.

Further, processing for evaluating the estimated pulse wave component output by the selecting unit 140 shown in FIG. 5 and discriminating whether noise removal processing by another body motion signal is necessary may be added. Specifically, when a fundamental frequency is present in an estimated pulse wave component extracted on the basis of a first body motion signal and a ratio of the fundamental frequency and a noise component excluding the fundamental frequency is equal to or larger than a predetermined value or a high correlation equal to or higher than a predetermined correlation degree is found in the determination by the correlation coefficient, the noise removal processing by the next body motion signal is not performed and a selected estimated pulse wave component is selected as an output signal.

Consequently, first, by inputting the body motion signal in order from acceleration in the axial direction with the largest motion, a main body motion noise component is removed from the biological signal. Subsequently, if it is determined by evaluating the output estimated pulse wave component that noise is sufficiently attenuated, in the estimated pulse wave component, noise is sufficiently attenuated even if the removal processing for the body motion signal in all the directions is not performed. Therefore, when an exercise state of the subject is exercise in a fixed axial direction, time for the estimated-pulse-wave-component extraction processing is saved and high-speed processing and power consumption can be suppressed. When the exercise state of the subject is exercise in a complex plurality of axial directions, it is possible to attenuate superimposed noise components of multiple axes by repeating processing for extracting the estimated pulse wave component for each of the axes.

Note that the body motion signal is not limited to the acceleration signal alone. The body motion signal may be a contact pressure displacement amount signal by the contact pressure sensor. The body motion signal is not limited to acceleration signal and only has to be a signal indicating a correlation with noise superimposed on the biological signal.

1. A biological-information processing apparatus comprising:
a biological-signal detecting unit that detects a biological signal including a pulse wave component and a body motion noise component;
a body-motion-signal detecting unit that detects a body motion signal; and
a body-motion-noise removing unit that separates the pulse wave component and the body motion noise component from the biological signal on the basis of the body motion signal, wherein
the body-motion-noise removing unit includes:
a plurality of filter units having different learning characteristics;
a correlation-information calculating unit that calculates correlation information indicating a correlation degree between the body motion signal and output signals from the plurality of filter units; and
a selecting unit that selects the output signal from the plurality of filter units on the basis of the correlation information.

2. The biological-information processing apparatus according to claim 1, wherein the learning characteristics include a step size for controlling a following characteristic to fluctuation in the body motion signal.

3. The biological-information processing apparatus according to claim 1, wherein
the correlation-information calculating unit calculates, for each of the output signals from the filter units, the correlation information on the basis of the body motion signal, and
the selecting unit selects the output signal from the filter unit in which an absolute value of the correlation information is smallest.

4. The biological-information processing apparatus according to claim 3, wherein the output signals from the filter units are estimated pulse wave signals for estimating the pulse wave component.

5. The biological-information processing apparatus according to claim 1, wherein
the correlation-information calculating unit calculates the correlation information on the basis of each of the output signals from the filter units and the body motion signal, and
the selecting unit selects the output signal from the filter unit in which an absolute value of the correlation information is largest.

6. The biological-information processing apparatus according to claim 5, wherein the output signals from the filter units are estimated body motion noise signals for estimating the body motion noise component.

7. The biological-information processing apparatus according to claims 1, wherein, when a difference between the correlation information of the filter unit that outputs the selected output signal and the correlation information of the other filter units exceeds a predetermined threshold, the selecting unit sets, as the learning characteristics of the other filters, the learning characteristics of the filter units that outputs the selected output signal.

8. The biological-information processing apparatus according to claims 1, wherein
the body motion signal includes acceleration signals in one axial direction or at least two axial directions crossing each other, and
the signals from the axes are sequentially applied as the body motion signal.

9. The biological-information processing apparatus according to claims 1, wherein the body motion signal includes a contact pressure signal indicating pressing of a detection part of the biological signal.

10. The biological-information processing apparatus according to claims 1, further comprising a control unit that calculates a pulse rate on the basis of the signal selected by the selecting unit.

11. A biological-information processing method comprising:
a biological-signal detecting step for detecting a biological signal including a pulse wave component and a body motion noise component;
a body-motion-signal detecting step for detecting a body motion signal;
a body-motion-noise removal processing step for separating the pulse wave component and the body motion noise component from the biological signal on the basis of the body motion signal using a plurality of filter steps for separating the pulse wave component and the body motion noise component, the plurality of filter steps having different learning characteristics;
a correlation-information calculating step for calculating correlation information indicating a correlation degree between the body motion signal and output signals from the plurality of filter steps; and
a selecting step for selecting the output signal from the plurality of filter units on the basis of the correlation information.
